(19) United States
(12) Patent Application Publication
(10) Pub. No.: US 2001/0052156 A1
(43) Pub. Date: Dec. 20, 2001

(54) DYES

(76) Inventors: ANDREAS JOACHIM BITTNER, OFFENBACH (DE); ASTRID KLEEN, ERKARTH (DE)

Correspondence Address: HENKEL CORPORATION, 2500 RENAISSANCE BLVD, STE 200, GULPH MILLS, PA, 19406, US

(21) Appl. No.: 09/214,281

(22) PCT Filed: Jul. 2, 1997
(86) PCT No.: PCT/EP97/03486

§ 371 Date: None

(30) Foreign Application Priority Data
Jul. 3, 1996 (DE) 196 26 683.1
Jul. 3, 1996 (DE) 196 26 721.8

(51) Int. Cl. A61K 7/13

(52) U.S. Cl. 8/405; 8/406; 8/408

(57) ABSTRACT

A colorant for coloring keratin fibers wherein the colorant contains a substantive dye which is a 4-nitroaniline derivative corresponding to formula (I):

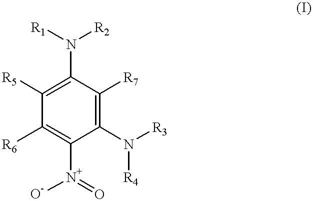

in which R₁, R₂, R₃ and R₄ independently of one another represent a hydrogen atom, an alkyl, hydroxyalkyl, alkoxyalkyl, carbamyl alkyl, mesylaminoalkyl, acetylaminoalkyl, ureidoalkyl, carbalkoxyaminoalkyl, sulfalkyl, piperidinoalkyl, morpholinoalkyl or phenyl radical optionally substituted by an amino group in the para position, the alkyl or alkoxy groups containing 1 to 4 carbon atoms, with the proviso that the four substituents R₁, R₂, R₃ and R₄ do not simultaneously represent hydrogen, and the groups —NR₁R₂ and —NR₃R₄ may also represent an aziridine, acetidine, pyrrolidine, piperidine, azepan, azocine, morpholine, thiomorpholine or piperazine ring which may also bear another substituent R₈ at the nitrogen atom, R₈ being a hydrogen atom, a (C₁₋₄)-alkyl, hydroxy-(C₂₋₃)-alkyl, (C₁₋₄)-alkoxy-(C₂₋₃)-alkyl, amino-(C₂₋₃)-alkyl or 2,3-dihydroxypropyl group, and R₅, R₆ and R₇ independently of one another represent ahydrogen atom, a halogen atom, a (C₁₋₄)-alkyl, (C₁₋₄)-alkoxy, carboxy, sulfo or (C₂₋₄)-hydroxyalkyl group, or a physiologically compatible salt of the compounds with an inorganic or organic acid.

This invention relates to colorants for coloring keratin fibers which contain special 4-nitroaniline derivatives as substantive dyes.

Besides so-called oxidation dyes, substantive dyes are also used for coloring keratin fibers. In contrast to oxidation dyes where the dye precursors penetrate into the hair and form the actual dyes thereon under the influence of oxidizing agents, substantive dyes are capable of directly coloring the hair. Under the boundary conditions prevailing in practice (low coloring temperature and short coloring time), they produce intensive colors with good fastness properties, the dyes diffusing into the fibers during the coloring process. Nitrophenylenediamines, nitroaminophenols, anthraquinones or indophenols are normally used as substantive dyes for coloring keratin fibers. Substantive dyes for obtaining various color tones play an important part in the coloring of hair. They should produce the required color with sufficient intensity during the coloring process. The colors obtained must be fast to light and acids. The dyes should not alter the original color of the hair under normal wearing conditions and, in addition, should be both dermatologically and toxicologically safe.

Large numbers of substantive dyes for coloring keratin fibers are already known. Red and yellow substantive dyes are disclosed, for example, in DE-AS 21 57 844 and in DE-OS 33 23 207. However, these known dyes have certain disadvantages. Thus, the solubility of the compounds in question, which is important for satisfactory coloring, is very poor. The luminosity and intensity of the colors obtained are also inadequate.

Accordingly, there is a constant need for new substantive hair dyes which are capable of satisfying increasingly more stringent requirements in regard to their fastness to light and to washing, their depth of color and their toxicological and dermatological properties. New substantive dyes in the yellow and red ranges of the color spectrum are required in particular for special yellow or Bordeaux tones.

Accordingly, the problem addressed by the present invention was to provide new compounds which would satisfy the requirements substantive dyes are expected to meet to a particular degree.

It has now surprisingly been found that the compounds corresponding to general formula (I) satisfy these requirements particularly well.

Accordingly, the present invention relates to colorants for color keratin fibers which contain as substantive dye a 4-nitroaniline derivative corresponding to formula (I):

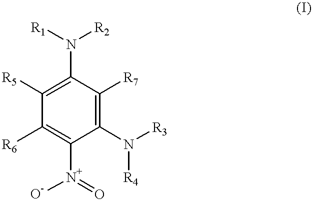

in which R₁, R₂, R₃ and R₄ independently of one another represent a hydrogen atom, an alkyl, hydroxyalkyl, alkoxyalkyl, carbamyl alkyl, mesylaminoalkyl, acetylaminoalkyl, ureidoalkyl, carbalkoxyaminoalkyl, sulfalkyl, piperidinoalkyl, morpholinoalkyl or phenyl radical optionally substituted by an amino group in the para position, these alkyl or alkoxy groups containing 1 to 4 carbon atoms, with the proviso that the substituents R₁, R₂, R₃ and R₄ do not simultaneously represent hydrogen, and the groups —NR₁R₂ and/or —NR₃R₄ may also stand for an aziridine, acetidine, pyrrolidine, piperidine, azepan, azocine, morpholine, thiomorpholine or piperazine ring which may also bear another substituent R₈ at the nitrogen atom, R₈ being a hydrogen atom, a (C₁₋₄)-alkyl, hydroxy-(C₂₋₃)-alkyl, (C₁₋₄)-alkoxy-(C₂₋₃)-alkyl, amino-(C₂₋₃)-alkyl or 2,3-dihydroxypropyl group, and R₅, R₆ and R₇ independently of one another represent a hydrogen atom, a halogen atom, a (C₁₋₄)-alkyl, (C₁₋₄)-alkoxy, carboxy, sulfo or (C₂₋₄)-hydroxyalkyl group,

or a physiologically compatible salt of these compounds with an inorganic or organic acid.

Keratin fibers in the context of the invention are pelts, wool, feathers and, in particular, human hair. Although the oxidation colorants according to the invention are primarily suitable for coloring keratin fibers, there is nothing in principle to stop them being used in other fields.

The compounds corresponding to formula (I) may be prepared by known methods. Reference is expressly made in this regard to the Examples of the present specification.

Particularly outstanding coloring properties are shown by compounds corresponding to formula (I) in which the group —NR₁R₂ or —NR₃R₄ stands for an aziridine, acetidine, pyrrolidine, piperidine, azepan, azocine, morpholine, thiomorpholine or piperazine ring which may even bear another substituent R₈ at the nitrogen atom, R₈ being a hydrogen atom, a (C₁₋₄)-alkyl, a hydroxy-(C₂₋₃)-alkyl, a (C₁₋₄)-alkoxy-(C₂₋₃)-alkyl, an amino-(C₂₋₃)-alkyl or a 2,3-dihydroxypropyl group.

Preferred substituents R₁ to R₇ where allowable within the scope of general formula (I) are hydrogen, methyl, ethyl, 2-hydroxyethyl, 2-aminoethyl and 2,3-dihydroxypropyl groups and, for R₅ to R₇, halogen atoms.

Other preferred compounds of formula (I) where R₁ and R₂ are not hydrogen are those in which either R₅ or R₇ stands for hydrogen. Other preferred compounds of formula (I) in which R₃ and R₄ are not hydrogen are those in which R₇ stands for hydrogen.

The compounds corresponding to formula (I) may be present both as free bases and in the form of their physiologically compatible salts with inorganic or organic acids, for example hydrochlorides, sulfates and hydrobromides. Other acids suitable for salt formation are phosphoric acid and also acetic acid, propionic acid, lactic acid and citric acid. Accordingly, the following observations on the compounds corresponding to formula (I) always apply to these salts also.

The hair colorants according to the invention contain the compounds corresponding to formula (I) in a quantity of preferably 0.001 to 10% by weight and, more preferably, 0.1 to 5% by weight, based on the oxidation colorant as a whole. Both here and in the following, the expression “colorant as a whole” refers to the product which is presented to the user. Depending upon the particular formulation, this product may be applied to the hair either directly or, if it additionally contains oxidation dye precursors, after mixing with water or, for example, an aqueous solution of an oxidizing agent.

In a preferred embodiment, the colorants according to the invention contain only the compounds of formula (I) as dye component.

However, the number of shades obtainable is distinctly increased if, in addition to the compounds of formula (I), the colorant also contains another substantive dye or an oxidation dye precursor.

In a second, likewise preferred embodiment, the colorants according to the invention contain at least one other substantive dye in addition to the compounds corresponding to formula (I) in claim 1 and, optionally, oxidation dye precursors for further modifying the color tones. The substantive dye in question belongs, for example, to the group consisting of nitrophenylenediamines, nitroaminophenols, anthraquinones or indophenols. Preferred substantive dyes are the compounds known under the International names or commercial names of HC Yellow 2, HC Yellow 4, Basic Yellow 57, Disperse Orange 3, HC Red 3, HC Red BN, Basic Red 76, HC Blue 2, Disperse Blue 3, Basic Blue 99, HC Violet 1, Disperse Violet 1, Disperse Violet 4, Disperse Black 9, Basic Brown 16, Basic Brown 17, picramic acid and Rodol R and also 4-amino-2-nitrodiphenylamine-2′-carboxylic acid, 6-nitro-1,2,3,4-tetrahydroquinoxaline, (N-2,3-dihydroxypropyl-2-nitro-4-trifluoromethyl)-aminobenzene and 4-N-ethyl-1,4-bis-(2′-hydroxyethylamino)-2-nitrobenzene hydrochloride. The colorants according to this embodiment of the invention contain the other substantive dye in a quantity of preferably 0.01 to 20% by weight, based on the colorant as a whole.

In addition, the colorants according to the invention may also contain naturally occurring dyes such as, for example, henna red, henna neutral, henna black, camomile blossom, sandalwood, black tea, black alder bark, sage, logwood, madder root, catechu, sedre and alkanet.

In a third preferred embodiment, therefore, the colorants according to the invention optionally contain at least one oxidation dye precursor of the primary intermediate type in addition to the compounds corresponding to formula (I) in claim 1 and, optionally, substantive dyes and an oxidation dye precursor of the secondary intermediate type.

According to the invention, preferred primary intermediates are p-phenylene diamine, p-toluylene diamine, p-aminophenol, 3-methyl-1,4-diaminobenzene, 1-(2′-hydroxyethyl)-2,5-diaminobenzene, N,N-bis-(2-hydroxyethyl)-p-phenylene diamine, 2-(2,5-diaminophenoxy)-ethanol, 1-phenyl-3-carboxyamido-4-amino-5-pyrazolone, 4-amino-3-methyl phenol, 2-methylamino-4-aminophenol, 2,4,5,6-tetraaminopyrimidine, 2-hydroxy-4,5,6-triaminopyrimidine, 4-hydroxy-2,5,6-triaminopyrimidine, 2,4-dihydroxy-5,6-diaminopyrimidine, 2-dimethylamino-4,5,6-triaminopyrimidine and 2-hydroxyethylaminomethyl-4-aminophenol.

According to the invention, p-toluylene diamine, p-aminophenol, 1-(2′-hydroxyethyl)-2,5-diaminobenzene, 4-amino-3-methylphenol, 2-methylamino-4-aminophenol and 2,4,5,6-tetraaminopyrimidine are most particularly preferred.

This embodiment does of course also encompass the use of several primary intermediates. According to the invention, preferred primary intermediate combinations are

p-toluylene diamine, p-phenylene diamine,

3-methyl-4-aminoaniline, p-toluylene diamine,

p-toluylene diamine, 4-amino-3-methylphenol,

p-toluylene diamine, 2-methylamino-4-aminophenol,

2,4,5,6-tetraaminopyrimidine, 1-(2′-hydroxyethyl)-2,5-diaminobenzene,

2,4,5,6-tetraaminopyrimidine, p-toluylene diamine.

In a fourth preferred embodiment, the colorants according to the invention optionally contain at least one oxidation dye precursor of the secondary intermediate type in addition to the compounds corresponding to formula (I) in claim 1 and, optionally, other substantive dyes and oxidation dye precursors of the primary intermediate type.

According to the invention, preferred secondary intermediates are 1-naphthol, pyrogallol, 1,5-, 2,7- and 1,7-dihydroxynaphthalene, o-aminophenol, 5-amino-2-methylphenol, m-aminophenol, resorcinol, resorcinol monomethyl ether, m-phenylene diamine, 1-phenyl-3-methyl-5-pyrazolone, 2,4-dichloro-3-aminophenol, 1,3-bis-(2,4-diaminophenoxy)-propane, 4-chlororesorcinol, 2-chloro-6-methyl-3-aminophenol, 2-methyl resorcinol, 5-methyl resorcinol, 2,5-dimethyl resorcinol, 2,6-dihydroxypyridine, 2,6-diaminopyridine, 2-amino-3-hydroxypyridine, 2,6-dihydroxy-3,4-diaminopyridine, 3-amino-2-methylamino-6-methoxypyridine, 4-amino-2-hydroxytoluene, 2,6-bis-(2-hydroxyethylamino)-toluene, 2,4-diaminophenoxyethanol, 2-amino-4-hydroxyethylaminoanisole.

According to the invention, 1,7-dihydroxynaphthalene, m-aminophenol, 2-methyl resorcinol, 4-amino-2-hydroxytoluene, 2-amino-4-hydroxyethylaminoanisole and 2,4-diaminophenoxyethanol are particularly preferred.

This embodiment does of course also encompass the use of several secondary intermediates. According to the invention, preferred secondary intermediate combinations are

resorcinol, m-phenylene diamine, 4-chlororesorcinol, 2-amino-4-hydroxyethylaminoanisole,

2-methyl resorcinol, 4-chlororesorcinol, 2-amino-3-hydroxypyridine,

resorcinol, m-aminoaniline, 2-hydroxy4-aminotoluene,

3-methyl-4-aminoaniline, m-aminoaniline, 2-hydroxy-4-aminotoluene, 2-amino-3-hydroxypyridine,

2-methyl resorcinol, m-aminoaniline, 2-hydroxy-4-aminotoluene, 2-amino-3-hydroxypyridine.

The primary and secondary intermediates are normally used in a substantially equimolar ratio to one another. Although it has proved to be useful to employ the primary and secondary intermediates in an equimolar ratio, a certain excess of individual oxidation dye precursors is by no means a disadvantage, so that the primary and secondary intermediates may advantageously be present in the colorant in a molar ratio of 1:0.5 to 1:2. The total quantity of oxidation dye precursors is generally at most 20% by weight, based on the colorant as a whole.

The substantive dyes compulsorily or optionally present or the oxidation dye precursors optionally present do not have to be single compounds. Instead, the hair colorants according to the invention—due to the processes used for producing the individual dyes—may contain small quantities of other components providing they do not adversely affect the coloring result or have to be ruled out for other reasons, for example toxicological reasons.

Typical formulations for the oxidation colorants according to the invention are preparations based on water or non-aqueous solvents and powders.

In one preferred embodiment for the production of the colorants according to the invention, the substantive dyes corresponding to formula (I) and optionally other dyes and oxidation dye precursors are incorporated in a suitable water-containing carrier. For coloring hair, such carriers are, for example, cremes, emulsions, gels or even surfactant-containing foaming solutions, for example shampoos, foam aerosols or other formulations suitable for application to the hair. The hair colorants according to the invention are adjusted to a pH value of preferably 6.5 to 11.5 and, more preferably, 9 to 10.

The colorants according to the invention may also contain any of the anionic surfactants suitable for use on the human body. Such substances are characterized by a water-solubilizing anionic group such as, for example, a carboxylate, sulfate, sulfonate or phosphate group and a lipophilic alkyl group containing around 10 to 22 carbon atoms. In addition, glycol or polyglycol ether groups, ether, amide and hydroxyl groups and—generally—ester groups may also be present in the molecule. The following are examples of suitable anionic surfactants—in the form of the sodium, potassium and ammonium salts and the mono-, di- and trialkanolammonium salts containing 2 or 3 carbon atoms in the alkanol group:

linear and branched fatty acids containing 8 to 22 carbon atoms (soaps),

ether carboxylic acids corresponding to the formula R—O—(CH₂—CH₂O)ₓ—CH₂—COOH, in which R is a linear alkyl group containing 10 to 22 carbon atoms and x=0 or 1 to 16,

acyl sarcosides containing 10 to 18 carbon atoms in the acyl group,

acyl taurides containing 10 to 18 carbon atoms in the acyl group,

acyl isethionates containing 10 to 18 carbon atoms in the acyl group,

sulfosuccinic acid mono- and dialkyl esters containing 8 to 18 carbon atoms in the alkyl group and sulfosuccinic acid monoalkyl polyoxyethyl esters containing 8 to 18 carbon atoms in the alkyl group and 1 to 6 oxyethyl groups,

linear alkane sulfonates containing 12 to 18 carbon atoms,

linear α-olefin sulfonates containing 12 to 18 carbon atoms,

α-sulfofatty acid methyl esters of fatty acids containing 12 to 18 carbon atoms,

alkyl sulfates and alkyl polyglycol ether sulfates corresponding to the formula R—O(CH₂—CH₂O)ₓ—SO₃H, in which R is a preferably linear alkyl group containing 10 to 18 carbon atoms and x=0 or 1 to 12,

mixtures of surface-active hydroxysulfonates according to DE-A-37 25 030,

sulfated hydroxyalkyl polyethylene and/or hydroxyalkylene propylene glycol ethers according to DE-A-37 23 354,

sulfonates of unsaturated fatty acids containing 12 to 24 carbon atoms and 1 to 6 double bonds according to DE-A-39 26 344,

esters of tartaric acid and citric acid with alcohols in the form of addition products of around 2 to 15 molecules of ethylene oxide and/or propylene oxide with fatty alcohols containing 8 to 22 carbon atoms.

Preferred anionic surfactants are alkyl sulfates, alkyl polyglycol ether sulfates and ether carboxylic acids containing 10 to 18 carbon atoms in the alkyl group and up to 12 glycol ether groups in the molecule and, in particular, salts of saturated and, more particularly, unsaturated C₈₋₂₂ carboxylic acids, such as oleic acid, stearic acid, isostearic acid and palmitic acid.

In the context of the invention, zwitterionic surfactants are surface-active compounds which contain at least one quaternary ammonium group and at least one —COO⁽⁻⁾ or —SO₃⁽⁻⁾ group in the molecule. Particularly suitable zwitterionic surfactants are the so-called betaines, such as N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. A preferred zwitterionic surfactant is the fatty acid amide derivative known by the CTFA name of Cocamidopropyl Betaine.

Ampholytic surfactants are surface-active compounds which, in addition to a C₈₋₁₈ alkyl or acyl group, contain at least one free amino group and at least one —COOH or —SO₃H group in the molecule and which are capable of forming inner salts. Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids containing around 8 to 18 carbon atoms in the alkyl group. Particularly preferred ampholytic surfactants are N-cocoalkyl aminopropionate, cocoacyl aminoethyl aminopropionate and C₁₂₋₁₈ acyl sarcosine.

Nonionic surfactants contain, for example, a polyol group, a polyalkylene glycol ether group or a combination of polyol and polyglycol ether groups as the hydrophilic group. Examples of such compounds are

products of the addition of 2 to 30 moles of ethylene oxide and/or 0 to 5 moles of propylene oxide to linear fatty alcohols containing 8 to 22 carbon atoms, to fatty acids containing 12 to 22 carbon atoms and to alkylphenols containing 8 to 15 carbon atoms in the alkyl group,

C₁₂₋₂₂ fatty acid monoesters and diesters of products of the addition of 1 to 30 moles of ethylene oxide to glycerol,

C₈₋₂₂ alkyl mono- and oligoglycosides and ethoxylated analogs thereof,

products of the addition of 5 to 60 moles of ethylene oxide to castor oil and hydrogenated castor oil,

products of the addition of ethylene oxide to sorbitan fatty acid esters,

products of the addition of ethylene oxide to fatty acid alkanolamides.

Examples of cationic surfactants suitable for use in the hair treatment formulations according to the invention are, in particular, quaternary ammonium compounds. Preferred quaternary ammonium compounds are ammonium halides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride. Other cationic surfactants suitable for use in accordance with the invention are the quaternized protein hydrolyzates.

Also suitable for use in accordance with the invention are cationic silicone oils such as, for example, the commercially available products Q2-7224 (manufacturer: Dow Corning; a stabilized trimethyl silyl amodimethicone), Dow Corning 929 Emulsion (containing a hydroxylamino-modified silicone which is also known as Amodimethicone), SM-2059 (manufacturer: General Electric), SLM-55067 (manufacturer: Wacker) and Abil®-Quat 3270 and 3272 (manufacturer: Th. Goldschmidt; diquaternary polydimethyl siloxanes, Quaternium-80).

Alkyl amidoamines, particularly fatty acid amidoamines, such as the stearyl amidopropyl dimethyl amine obtainable as Tego Amid®S 18, are distinguished not only by their favorable conditioning effect, but also and in particular by their ready biodegradability.

Quaternary ester compounds, so-called “esterquats”, such as the dialkyl ammonium methosulfates and methyl hydroxyalkyl dialkoyloxyalkyl ammonium methosulfates marketed under the trade name of Stepantex®), are also readily biodegradable.

One example of a quaternary sugar derivative suitable for use as a cationic surfactant is the commercially available product Glucquat®)100 (CTFA name: Lauryl Methyl Gluceth-10 Hydroxypropyl Dimonium Chloride).

The compounds containing alkyl groups used as surfactants may be single compounds. In general, however, these compounds are produced from native vegetable or animal raw materials so that mixtures with different alkyl chain lengths dependent upon the particular raw material are obtained.

The surfactants representing addition products of ethylene and/or propylene oxide with fatty alcohols or derivatives of these addition products may be both products with a “normal” homolog distribution and products with a narrow homolog distribution. Products with a “normal” homolog distribution are mixtures of homologs which are obtained in the reaction of fatty alcohol and alkylene oxide using alkali metals, alkali metal hydroxides or alkali metal alcoholates as catalysts. By contrast, narrow homolog distributions are obtained when, for example, hydrotalcites, alkaline earth metal salts of ether carboxylic acids, alkaline earth metal oxides, hydroxides or alcoholates are used as catalysts. The use of products with a narrow homolog distribution can be of advantage.

Other active substances, auxiliaries and additives are, for example,

nonionic polymers such as, for example, vinyl pyrrolidone/vinyl acrylate copolymers, polyvinyl pyrrolidone and vinyl pyrrolidone/vinyl acetate copolymers and polysiloxanes,

cationic polymers, such as quaternized cellulose ethers, polysiloxanes containing quaternary groups, dimethyl diallyl ammonium chloride polymers, acrylamide/dimethyl diallyl ammonium chloride copolymers, dimethyl aminoethyl methacrylate/vinyl pyrrolidone copolymers quaternized with diethyl sulfate, vinyl pyrrolidone/imidazolinium methochloride copolymers and quaternized polyvinyl alcohol,

zwitterionic and amphoteric polymers such as, for example, acrylamidopropyl/trimethyl ammonium chloride/acrylate copolymers and octyl acrylamide/methyl methacrylate/tert.butyl aminoethyl methacrylate/2-hydroxypropyl methacrylate copolymers,

anionic polymers such as, for example, polyacrylic acids, crosslinked polyacrylic acids, vinyl acetate/crotonic acid copolymers, vinyl pyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert.butyl acrylamide terpolymers,

thickeners, such as agar agar, guar gum, alginates, xanthan gum, gum arabic, karaya gum, carob bean flour, linseed gums, dextrans, cellulose derivatives, for example methyl cellulose, hydroxyalkyl cellulose and carboxymethyl cellulose, starch fractions and derivatives, such as amylose, amylopectin and dextrins, clays such as, for example, bentonite or fully synthetic hydrocolloids such as, for example, polyvinyl alcohol,

structurants, such as glucose, maleic acid and lactic acid,

hair-conditioning compounds, such as phospholipids, for example soya lecithin, egg lecithin and kephalins, and also silicone oils,

protein hydrolyzates, more particularly elastin, collagen, keratin, milk protein, soya protein and wheat protein hydrolyzates, condensation products thereof with fatty acids and quaternized protein hydrolyzates,

perfume oils, dimethyl isosorbide and cyclodextrins,

solubilizers, such as ethanol, isopropanol, ethylene glycol, propylene glycol, glycerol and diethylene glycol,

antidandruff agents, such as Piroctone Olamine and Zinc Omadine,

alkalizing agents such as, for example, ammonia, monoethanolamine, 2-amino-2-methylpropanol and 2-amino-2-methylpropane-1,3-diol,

other substances for adjusting the pH value,

active substances, such as panthenol, pantothenic acid, allantoin, pyrrolidone carboxylic acids and salts thereof, plant extracts and vitamins,

cholesterol,

UV filters,

consistency promoters, such as sugar esters, polyol esters or polyol alkyl ethers,

fats and waxes, such as spermaceti, beeswax, montan wax, paraffins, fatty alcohols and fatty acid esters,

fatty acid alkanolamides,

complexing agents, such as EDTA, NTA and phosphonic acids,

swelling and penetration agents, such as glycerol, propylene glycol monoethyl ether, carbonates, hydrogen carbonates, guanidines, ureas and primary, secondary and tertiary phosphates,

opacifiers, such as latex,

pearlescers, such as ethylene glycol mono- and distearate,

propellents, such as propane/butane mixtures, N₂O, dimethyl ether, CO₂ and air and

antioxidants.

To produce the colorants according to the invention, the constituents of the water-containing carrier are used in the usual quantities for this purpose. For example, emulsifiers are used in concentrations of 0.5 to 30% by weight while thickeners are used in concentrations of 0.1 to 25% by weight, based on the colorant as a whole.

If the colorants according to the invention contain oxidation dye precursors, the oxidative development of the color may be carried out in principle with atmospheric oxygen or with an oxidizing agent present in or added to the colorant immediately before application.

In a first preferred embodiment, a chemical oxidizing agent is used. This is particularly advantageous in cases where human hair is to be not only colored, but also lightened. Particularly suitable oxidizing agents are hydrogen peroxide or addition products thereof with urea, melamine or alkali metal borate. In a particularly preferred variant of this embodiment, the colorant according to the invention is mixed immediately before application with the preparation of an oxidizing agent, more particularly an aqueous H₂O₂ solution. The ready-to-use hair coloring preparation formed should preferably have a pH value of 6 to 10. In a particularly preferred embodiment, the hair colorant is used in a mildly alkaline medium. The application temperatures may be in the range from 15 to 40° C. After a contact time of about 30 minutes, the hair colorant is removed from the hair to be colored by rinsing. There is no need for the hair to be washed with a shampoo where a carrier of high surfactant content, for example a coloring shampoo, has been used.

In the particular case of hair which is difficult to color, the preparation containing the oxidation dye precursors may be applied to the hair without preliminary mixing with the oxidation component. The oxidation component is applied after a contact time of 20 to 30 minutes, optionally after rinsing. After another contact time of 10 to 20 minutes, the hair is rinsed and, if desired, shampooed.

In a second embodiment, the color is developed with atmospheric oxygen. In this case, it is of advantage to add an oxidation catalyst to the colorant according to the invention. Suitable oxidation catalysts are metal salts and metal complexes, transition metals being preferable. Copper, manganese, cobalt, selenium, molybdenum, bismuth and ruthenium compounds are preferred. Copper(II) chloride, sulfate and acetate can be preferred oxidation catalysts. Preferred metal complexes include the complexes with ammonia, ethylenediamine, phenanthroline, triphenyl phosphine, 1,2-diphenyl phosphinoethane, 1,3-diphenyl phosphinopropane or amino acids. The same colorant may of course also contain several oxidation catalysts. Particulars of the production of suitable catalysts can be found in the corresponding disclosure of EP 0 709 365 A1 (page 4, lines 19 to 42) to which reference is expressly made.

The oxidation may also be carried out with enzymes. In this case, the enzymes may be used both to produce oxidizing per compounds and to enhance the effect of an oxidizing agent present in small quantities. One example of an enzymatic process is the procedure where the effect of small quantities (for example 1% and less, based on the colorant as a whole) of hydrogen peroxide is enhanced by peroxidases.

The present invention also relates to the use of 4-nitroanilines corresponding to general formula (I) in claim 1 for coloring keratin fibers.

The compounds corresponding to general formula (I) may be converted by reduction of the 4-nitro group into compounds corresponding to general formula (Ia) in which the 4-nitro group is replaced by a 4-amino group:

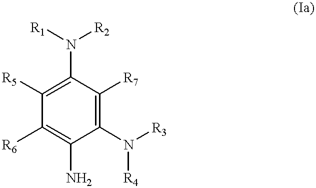

and R₁ to R₇ have the same meanings as in general formula (I). In the compounds corresponding to general formula (Ia), the 4-amino group may be converted by alkylation or alkoxylation into a substituted 4-amino group:

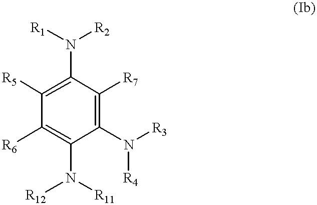

so that compounds corresponding to general formula (Ib) in which R₁ to R₇ have the meanings defined for general formula (I) and R₁₁ and R₁₂ independently of one another can represent a hydrogen atom, a (C₁₋₄)-alkyl, hydroxy-(C₂₋₃)-alkyl, alkoxy-(C₂₋₃)-alkyl, amino-(C₂₋₃)-alkyl or 2,3-dihydroxypropyl group.

These compounds represent oxidation dye precursors which have particularly outstanding coloring properties and, at the same time, secondary and primary intermediate properties.

### EXAMPLES

1. General Production Processes

1.1a. Production Processes Starting From 2-Nitroanilines

Compounds according to the invention corresponding to general formula (I), in which R₅, R₆ and R₇ are hydrogen atoms, may be prepared by reacting 2-nitro-5-haloacetanilides corresponding to general formula (II), where X—fluorine, chlorine, bromine or iodine, with amines corresponding to general formula (III), in which R₁ and R₂ are as defined in claim 1, in an alkaline reaction medium, optionally in the presence of phase transfer catalysts, to form correspondingly substituted 2-nitro-5-aminoanilines corresponding to general formula (IV). This reaction may optionally be carried out under pressure in an autoclave where the boiling point of the amine is lower than the reaction temperature or the reaction is otherwise incomplete. The compounds corresponding to formula (IV) are hydrolyzed and optionally converted by alkylation or alkoxylation into the compounds of general formula (I). The compounds corresponding to general formula (III) are standard chemical starting materials and may be commercially obtained. Suitable phase transfer catalysts are, for example, methyl or benzyl tri(C₆₋₈)-alkyl ammonium chloride.

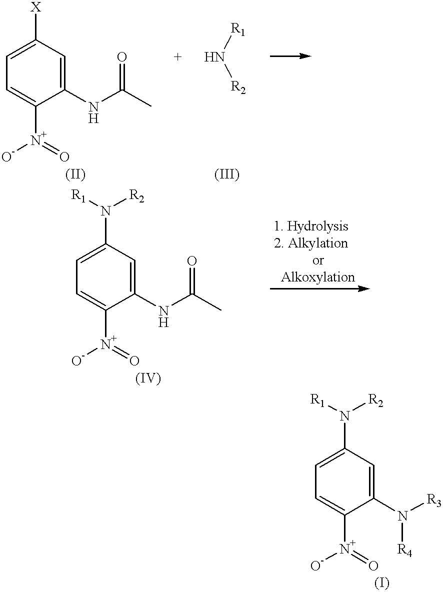

In a second process, compounds corresponding to general formula (I) may be obtained by reacting 5-halo-2-nitranilines corresponding to general formula (V), where R₃ and R₄ are as defined above, with amines corresponding to general formula (III) in the presence of phase transfer catalysts to form compounds corresponding to general formula (I). This reaction also may optionally be carried out under pressure in an autoclave if the boiling point of the amine is lower than the reaction temperature or the reaction is otherwise incomplete.

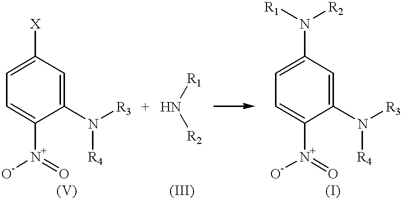

1.1 b. Production Processes Starting From 4-Nitroanilines

Compounds according to the invention corresponding to general formula (I), in which R₅, R₆ and R₇ are hydrogen atoms, may also be prepared by reacting 2-nitro-5-acetylaminohalobenzenes corresponding to general formula (II)′, where X=fluorine, chlorine, bromine or iodine, with amines corresponding to general formula (III)′, where R₃ and R₄ are as defined above, in an alkaline-reaction medium, optionally in the presence of phase transfer catalysts, to form optionally substituted 2-nitro-5-acetylaminoanilines corresponding to general formula (IV)′. This reaction may optionally be carried out under pressure in an autoclave where the boiling point of the amine is lower than the reaction temperature or the reaction is otherwise incomplete.

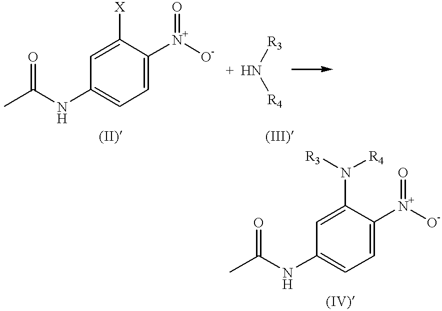

The compounds corresponding to formula (IV)′ are hydrolyzed to the compounds corresponding to general formula (V) and optionally converted by alkylation or alkoxylation into the compounds according to the invention corresponding to general formula (I). The compounds corresponding to general formula (III)′ are standard chemical starting materials and may be commercially obtained. Suitable phase transfer catalysts are, for example, methyl or benzyl tri(C₆₋₈)alkyl ammonium chloride.

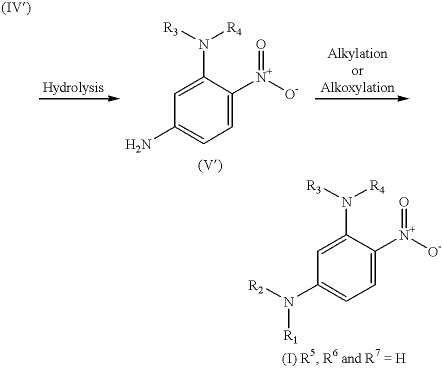

In a second process, compounds according to the invention corresponding to general formula (I) are prepared by reacting substituted 2-nitro-4-aminohalobenzenes corresponding to general formula (VI)′, in which X=fluorine, chlorine, bromine or iodine and R₃ and R₄ are as defined above, with amines corresponding to general formula (III)′, where R₁ and R₂ are as defined above, in an alkaline reaction medium, optionally in the presence of phase transfer catalysts, to form substituted 2-nitro-4-aminoanilines corresponding to general formula (I). This reaction also may optionally be carried out under pressure in an autoclave if the boiling point of the amine is lower than the reaction temperature or the reaction is otherwise incomplete.

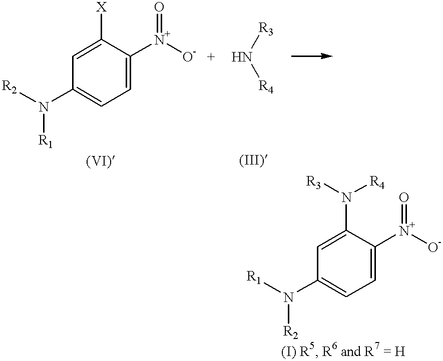

For alkoxylation, the compounds corresponding to general formula (V)′, in which R₁ and R₂=hydrogen, are converted into compounds corresponding to general formula (VIII)′ by reaction in an inert solvent with chloroformic acid-2-chloroethyl ester (VII) (R₉=CH₂CH₂Cl) or chloroformic acid-3-chloropropyl ester (VII) (R₉=CH₂CH₂CH₂Cl).

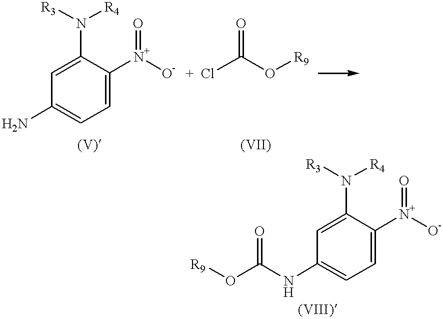

The intermediate products corresponding to general formula (VIII)′ are then reacted with strong bases, for example sodium or potassium hydroxide, to form compounds corresponding to general formula (IX)′, where R₁₀=CH₂CH₂OH) or CH₂CH₂CH₂OH, which react with known alkylating or alkoxylating agents

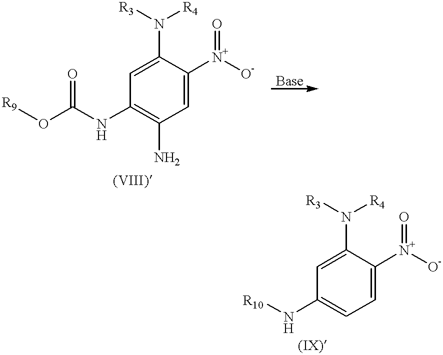

to form compounds corresponding to general formula (I), in which R₁ to R₄ are as already defined, and the compounds of general formula (I) obtained are optionally converted into salt form with an inorganic or organic acid.

In a third process, compounds according to the invention corresponding to general formula (I) may be obtained by reacting 3-halo-4-nitranilines corresponding to general formula (VI) with amines corresponding to general formula (IIIa), in which R₁ to R₄ are as defined above, optionally in the presence of phase transfer catalysts, to form compounds corresponding to general formula (I):

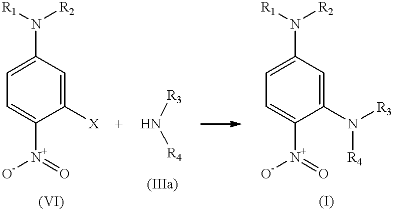

after the function X has been exchanged for NR₁R₂ or NR₃R₄ and, optionally, further alkylation or alkoxylation, compounds according to the invention corresponding to general formula (I) are obtained and are optionally converted into salt form with an inorganic or organic acid.

1.2. General Observations on the Production Processes

The first stage of these processes essentially comprises exchanging a halogen substituent for an amine substituent at the phenyl ring. The known processes are normally carried out with an excess of amine of about 40 to 80%. The products are obtained in yields of about 90% and with a purity of 95 to 96%. It has now surprisingly been found that higher yields can be obtained for the same or better purities and a faster conversion if the excess of piperazine is 30% or less, more particularly 5 to 10 mole-%, based on the quantities of compound (II), (VI) or (VIa) used. The reaction of the piperazine (III) with the compounds (II), (VI) or (VIa) is preferably carried out in the presence of alkali metal carbonates as acid-binding agents. In another preferred embodiment, the reaction is carried out in an organic solvent. Moreover, the reaction is preferably carried out under a pressure of 1 to 15 bar, more preferably under a pressure of 1 to 8 bar and most preferably under a pressure of 1 to 2.5 bar.

Suitable phase transfer catalysts are, for example, methyl or benzyl tri(C₆₋₈) alkyl ammonium chloride or tetraphenyl phosphinium chloride.

Suitable inorganic acids for salt formation are, for example, hydrochloric acid, sulfuric acid or phosphoric acid while suitable organic acids for salt formation are acetic acid, propionic acid, lactic acid or citric acid.

For alkoxylation, the compounds corresponding to general formula (I), in which R₃ and R₄=hydrogen, are converted into compounds corresponding to formula (VIII) in known manner by reaction in an inert solvent with chloroformic acid-2-chloroethyl ester (R₉=CH₂CH₂Cl) or chloroformic acid-3-chloropropyl ester (VII) (R₉=CH₂CH₂CH₂Cl).

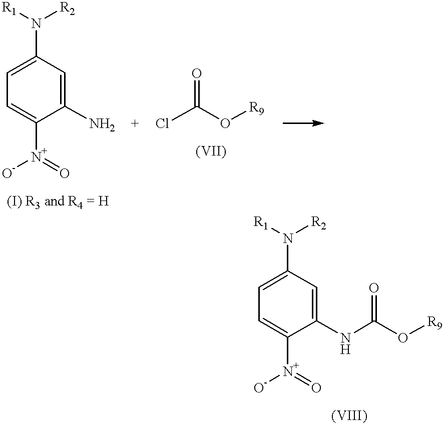

The intermediate products corresponding to formula (VIII) are then reacted with strong bases, for example sodium or potassium hydroxide, to form compounds corresponding to general formula (IX):

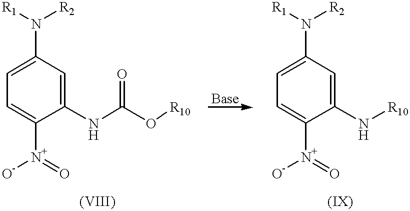

in which R₁₀=CH₂CH₂OH or CH₂CH₂CH₂OH,

which react with known alkylating or alkoxylating agents to form compounds corresponding to general formula (I), in which R₁ to R₄ are as already defined, and the compounds corresponding to general formula (I) obtained are optionally converted into salt form with an inorganic or organic acid.

1.3. Preparation of Special Compounds Corresponding to Formula (I)

The compounds prepared were characterized by IR spectra or IR(KBr pellet) and/or ¹H-NMR spectra (in D₆-DMSO). Where the spectra are shown in the following, only the very strong and strong bands are mentioned in the case of the IR spectra. In the data on the ¹H-NMR spectra, s=singlet, d=doublet, dd=doublet of the doublet, t=triplet, q=quartet, qi - quintet, m=multiplet, ³J and ⁴J=the couplings via three or four bonds and H³, H⁴, H⁵ and H⁶=the hydrogen atoms in positions 3, 4, 5 and 6 of the benzene rings.

1.3.1. Preparation of N, N-Dimethyl-3-Amino-4-Nitroaniline

Step a) N,N-Dimethyl-4-Nitro-3-Acetaminoaniline

100 ml of 1,2-dimethoxyethane, 21.5 g (0.1 mole) of 5-chloro-2-nitroacetanilide, 20.7 g (0.15 mole) of potassium carbonate and 16.9 g (0.15 mole) of 40% dimethylamine solution were introduced into an autoclave. The mixture was heated with stirring for 8 hours to 120° C., the inorganic salts were filtered off from the hot mixture and the solvent was removed in vacuo. The yellow powder precipitated was recrystallized from ethanol.

Yield: 21.3 g (95.4% of the theoretical)

IR: 3313 cm⁻¹ (v CH_{Ar}), 3313, 3143, 2927 cm⁻¹ (v CH), 1702 cm⁻¹ (v C═O), 1622 cm⁻¹(v C═C), 1575, 1548 cm⁻¹ (Vₐₛ NO₂) 1344, 1310 cm⁻¹ (vₛ NO₂).

¹H-NMR: 10.59 ppm (1H, s, NHAc); 8.02 ppm (H⁵, d, ³J_{H,H}=9.70 Hz); 7.63 ppm (H², d, ⁴J_{H,H}=2.71 Hz); 6.54 ppm (H⁶, dd, ³J_{H,H}=9.57 Hz, ⁴J_{H.H}=2.55 Hz); 3.07 ppm (6H, s, N(CH₃)₂); 2.17 ppm (3H, s, C(═O)CH₃).

Step b) N,N-Dimethyl-3-Amino-4-Nitroaniline

20.3 g (0.091 mole) of N,N-dimethyl-4-nitro-3-acetaminoaniline (from step a) were introduced into a mixture of 75 ml of water and 21 ml of methanol, followed by refluxing for 1 hour with 20.7 ml of concentrated hydrochloric acid. On completion of the reaction, the mixture was adjusted to pH 7 with aqueous ammonia and the product was precipitated by stirring, filtered off under suction and washed with water.

Yield: 15.9 g (96.4% of the theoretical)

IR: 3468, 3348 cm⁻¹ (v CH_{Ar}), 2922 cm⁻¹ (v CH), 1619 cm⁻¹ (v C═C), 1557 cm⁻¹ (vₐₛ NO₂), 1312 cm⁻¹ (vₛ NO₂).

¹H-NMR: 7.82 ppm (H⁵, d, ³J_{H,H}=9.75 Hz); 7.25 ppm (2H, s, NH₂); 6.20 ppm (H⁶, dd, ³J_{H,H}=9.81 Hz, ⁴J_{H,H}=2.68 Hz); 5.96 ppm (H², d, ⁴J_{H,H}=2.67 Hz); 3.00 ppm (6H, s, N(CH₃)₂).

1.3.2. Preparation of N-(4-Nitro-3-Aminophenyl)Morpholine

Step a) N-(4-Nitro-3-Acetaminophenyl)Morpholine

Step a) was carried out in the same way as Example 1.3.1. step a) by reacting 5-chloro-2-nitroacetanilide with morpholine.

Yield: 24.1 g (90.8% of the theoretical) yellow crystals

IR: 3438 cm⁻¹ (v CH_{Ar}), 3312, 3140, 2973, 2855 cm⁻¹ (v CH), 1698 cm⁻¹ (v C═O), 1617 cm⁻¹ (v C═C), 1580 cm⁻¹ (vₐₛ NO₂), 1312 cm⁻¹ (vₛ NO₂).

¹H-NMR: 10.43 ppm (1H, s, NHAc); 7.98 ppm (H⁵, d, ³J_{H,H}=9.55 Hz); 7.70 ppm (H, d, ⁴J_{H,H}=2.69 Hz); 6.78 ppm (H⁶, dd, ³J_{H,H}=9.59 Hz, ⁴J_{H,H}=2.72 Hz); 3.73 ppm (4H, t, ³J_{H,H}=4.85 Hz, N—(CH₂CH₂)₂O); 3.35 ppm (4H, t, ³J_{H,H}=4.91 (Hz, N(CH₃CH₂)₂O); 2.15 ppm (3H, s, C(═O)CH.).

Step b) N-(4-Nitro-3-Aminophenyl)Morpholine

Step b) was carried out in the same way as Example 1.3.1. step b) by reacting N-(4-nitro-3-acetaminophenyl)morpholine with hydrochloric acid.

Yield: 15.3 g (84.4% of the theoretical)

1.3.3. Preparation of N-Methyl-2-Nitro-5-Aminoaniline

Step a) N-Methyl-2-Nitro-5-Acetaminoaniline

100 ml of 1,2-dimethoxyethane, 21.5 g (0.1 mole) of 3-chloro-4-nitroacetanilide, 15.1 g (0.11 mole) of potassium carbonate and 12.4 g (0.11 mole) of 40% methylamine solution were introduced into an autoclave. The mixture was then heated with stirring for 8 hours to 120° C., the inorganic salts were filtered off from the hot mixture and the solvent was removed in vacuo. The yellow powder precipitated was recrystallized from ethanol.

Yield: 17 g (100% of the theoretical)

IR: 3351 cm⁻¹ (v CH_{Ar}), 3303, 3183, 2808 cm⁻¹ (v CH), 1694 cm⁻¹ (v C═O), 1639 cm⁻¹ (v C═C), 1582, 1562 cm⁻¹ (vₐₛ NO₂), 1366, 1325 cm⁻¹ (vₛ NO₂).

¹H-NMR: 10.2 ppm (1 H, s, NHAc); 8.43 ppm (1 H, m, NHCH₃); 8.19 ppm (H³, d, ³J_{H,H}=9.34 Hz); 7.56 ppm (H⁶, d,⁴J_{H,H}=2.95 Hz); 6.97 ppm (H⁴, dd, ³J_{H,H}=9.42 Hz, ⁴J_{H,H}=2.14 Hz); 3.08 ppm (3H, t, ³J_{H,H}=4.93 Hz, NCH₃); 2.27 ppm (3H, s, C(═O)CH₃).

Step b) N-Methyl-2-Nitro-5-Aminoaniline

15.9 g (0.095 mole) of 3-methylamino-4-nitroacetanilide (from step a) were introduced into a mixture of 78 ml of water and 26 ml of methanol, followed by refluxing for 1 hour with 25.8 ml of concentrated hydrochloric acid. On completion of the reaction, the mixture was adjusted to pH 7 with aqueous ammonia, the product was precipitated by stirring, filtered off under suction and washed with water.

Yield: 12.4 g (78.1 % of the theoretical)

IR: 3425 cm⁻¹ (v CH_{Ar}), 3336, 3227, 2926 cm⁻¹ (v CH), 1636 cm⁻¹ (v C═C), 1577 cm⁻¹ (vₐₛ NO₂), 1331 cm (vₛ NO₂).

¹H-NMR: 8.33 ppm (1 H, m, NHCH₃); 7.82 ppm (H³, d, ³J_{H,H}=9.37 Hz); 6.59 ppm (2H, s, NH₂); 6.0 ppm (H⁴, dd, ³J_{H,H}=9.36 Hz, ⁴J_{H,H}=2.12 Hz); 5.8 ppm (H⁶, d, ⁴J_{H,H}=2.11 Hz); 2.86 ppm (3H, t, ³J_{H,H}=4.96 Hz, NCH₃).

1.3.4. Preparation of N-Ethyl-2-Nitro-5-Aminoaniline

Step a) N-Ethyl-2-Nitro-5-Acetaminoaniline

Step a) was carried out in the same way as Example 1.3.3. step a) by reacting 3-chloro-4-nitroacetanilide with ethylamine.

Yield: 19.7 g (88.2% of the theoretical) yellow crystals

IR: 3343 cm⁻¹ (v CH_{Ar}), 3225, 2971, 2873 cm⁻¹ (v CH), 1702 cm⁻¹ (v C═O), 1621 cm⁻¹ (v C═C), 1582 cm⁻¹ (vₐₛ NO₂), 1367 cm⁻¹ (vₛ NO₂).

¹H-NMR: 10.31 ppm (1H, s, NHAc); 8.19 ppm (1H, t, NHEt, ³J_{H,H}=5.22 Hz); 8.02 ppm (H³, d, ³J_{H,H}=9.42 Hz); 7.46 ppm (H⁶, d, ⁴J_{H,H}=1.97 Hz); 6.79 ppm (H⁴, dd, ³J_{H,H}=9.39 Hz, ⁴J_{H,H}=2.01 Hz); 3.08 ppm (2H, m, ³J_{H,CH3}=5.71 Hz, ³J_{CH2,CH3}=6.99 Hz, NCH₂); 1.25 ppm (3H, t, ³J_{H,H}=7.11 Hz, NCH₂CH₃).

Step b) N-Ethyl-2-Nitro-5-Aminoaniline

Step b) was carried out in the same way as Example 1.3.3. step b) by reacting N-ethyl-2-nitro-5-acetaminoaniline with hydrochloric acid.

Yield: 15.3 g (84.4% of the theoretical)

IR: 3438 cm⁻¹ (v CH_{Ar}), 3339, 3232, 2979 cm⁻¹ (v CH), 1619 cm⁻¹ (v C═C), 1564 cm (vₐₛ NO₂), 1354 cm (vₛ NO₂).

¹H-NMR: 8.29 ppm (1 H, s, NH); 7.82 ppm (H³, d, ³J_{H,H}=9.22 Hz); 6.59 ppm (2H, s, NH₂); 6.0 ppm (H⁴, dd, ³J_{H,H}=9.48 Hz, ⁴J_{H,H}=2.0 Hz); 5.84 ppm (H⁶, d, ⁴J_{H,H}=2.03 Hz); 3.2 ppm (2H, m, ³J_{H,CH3}=5.45 Hz, ³J_{CH2,CH3}=7.08 Hz, NCH₂); 1.24 ppm (3H, t, ³J_{CH2,CH3}=7.12 Hz, NCH₂CH₃).

1.3.5. Preparation of N-n-Propyl-2-Nitro-5-Aminoaniline

Step a) N-n-Propyl-2-Nitro-5-Acetylaminoaniline

Step a) was carried out in the same way as Example 1.3.3. step a) by reacting 3-chloro-4-nitroacetanilide with n-propylamine.

Yield: 15.5 g (65.3% of the theoretical) yellow crystals

IR: 3349 cm⁻¹ (v CH_{Ar}), 3235, 2964, 2934 cm⁻¹ (v CH), 1694 cm⁻¹ (v C═O), 1623 cm⁻¹ (v C═C), 1582 cm⁻¹ (vₐₛ NO₂), 1326 cm⁻¹ (vₛ NO₂). ¹H-NMR: 10.30 ppm (1 H, s, NHAc); 8.25 ppm (1 H, t, NHPr,³J_{H,H}=5.40 Hz); 8.02 ppm (H³, d, ³J_{H,H}=9.35 Hz); 7.46 ppm (H⁶, d, ⁴J_{H,H}=2.08 Hz); 6.78 ppm (H⁴, dd, ³J_{H,H}=9.39 Hz, ⁴J_{H,H}=2.14 Hz); 3.22 ppm (2H, m, NCH₂); 2.09 ppm (3H, s, C(═O)CH₃); 1.65 ppm (2H, m, NCH₂CH₂CH₃); 0.96 ppm (3H, t, ³J_{H,H}=7.40 Hz, NCH₂CH₂CH₃).

Step b) N-n-propyl-2-Nitro-5-Aminoaniline

Step b) was carried out in the same way as Example 1.3.3. step b) by reacting N-n-propyl-2-nitro-5-acetaminoaniline with hydrochloric acid.

Yield: 11.7 g (98.3% of the theoretical)

IR: 3444 cm⁻¹ (v CH_{Ar}), 3343, 3232, 2964 cm⁻¹ (v CH), 1631 cm⁻¹ (v C═C), 1570 cm⁻¹ (vₐₛ NO₂), 1364 cm⁻¹ (vₛ NO₂).

¹H-NMR: 8.38 ppm (1H, t,³J_{H,H}=4.67 Hz, NH); 7.84 ppm (H³, d, ³J_{H,H}=9.36 Hz); 6.59 ppm (2H, s, NH₂); 6.02 ppm (H⁴, dd, ³J_{H,H}=9.38 Hz, ⁴J_{H,H}=1.81 Hz); 5.86 ppm (H⁶, s); 3.16 ppm (2H, m, ³J_{H,H}=7.21 Hz, NCH₂); 1.64 ppm (2H, t, J³_{H,H}=7.37 Hz, NCH₂CH₂CH₃); 0.97 ppm (3H, t, ³J_{H,H}=7.37 Hz, NCH₂CH₂-CH₃).

1.3.6. Preparation of N-iso-Butyl-2-Nitro-5-Aminoaniline

Step a) N-iso-Butyl-2-Nitro-5-Acetylaminoaniline

Step a) was carried out in the same way as Example 1.3.3. step a) by reacting 3-chloro-4-nitroacetanilide with isobutylamine.

Yield: 14.5 g (57.7% of the theoretical) yellow crystals

IR: 3347 cm⁻¹ (v CH_{Ar}), 3083, 2969, 2936 cm⁻¹ (v CH), 1704, 1680 cm⁻¹ (v C═O), 1621 cm⁻¹ (v C═C), 1581 cm⁻¹ (vₐₛ NO₂), 1325 cm⁻¹ (vₛ NO₂).

¹H-NMR: 10.31 ppm (1 H, s, NHAc); 8.09 ppm (1 H, t, NHBu, ³J_{H,H}=7.51 Hz); 8.08 ppm (H³, d, ³J_{H,H}=9.38 Hz); 7.53 ppm (H⁶, d, ⁴J_{H,H}=2.01 Hz); 6.78 ppm (H⁴, dd, ³J_{H,H}=9.38 Hz, ⁴J_{H,H}=2.09 Hz); 3.53 ppm (1H, qi, CH(CH₃)₂); 2.1 ppm (3H, s, C(═O)CH₃); 1.61 ppm (2H, m, ³J_{H,H}=7.09 Hz, NCH₂(CH₃)₂); 1.24 ppm (3H, d, ³J_{H,H}=6.34 Hz, syn-CH(CH₃)₂); 0.94 ppm (3H, t, ³J_{H,H}=7.42 Hz, anti-CH(CH₃)₂).

Step b) N-iso-Butyl-2-Nitro-5-Aminoaniline

Step b) was carried out in the same way as Example 1.3.3. step b) by reacting N-iso-butyl-2-nitro-5-acetaminoaniline with hydrochloric acid.

Yield: 11.5 g (55.2% of the theoretical)

Melting point: (red oil)

IR: 3467, 3350, 3234 cm⁻¹ (v CH_{Ar}), 2967, 2932, 2876 cm⁻¹ (v CH), 1631 cm⁻¹ (v C═C), 1568 cm⁻¹ (vₐₛ NO₂), 1355 cm⁻¹ (vₛ NO₂).

¹H-NMR: 8.33 ppm (1H, d, ³J_{H,H}=7.56 Hz, NH); 7.82 ppm (H³, d, ³J_{H,H}=9.46 Hz); 6.00 ppm (H⁴, dd, ³J_{H,H}=9.48 Hz, ⁴J_{H,H}=2.18 Hz); 5.87 ppm (H⁶, d, ⁴J_{H,H}=2.11 Hz); 3.49 ppm (1H, m, ³J_{H,H}=6.56 Hz, NCH₂CH); 1.59 ppm (2H, m,³J_{H,H}=9.21 Hz, NCH₂—CH(CH₃)₂); 1.21 ppm (3H, d, ³J_{H,H}=6.33 Hz, anti-NCH₂PH—(CH₃)₂); 0.93 ppm (3H, t, ³J_{H,H}=7.44 Hz, syn-NCH₂CH(CH₃)₂).

1.3.7. Preparation of N,N-Dimethyl-2-Nitro-5-Aminoaniline

Step a) N,N-Dimethyl-2-Nitro-5-Acetylaminoaniline

Step a) was carried out in the same way as Example 1.3.3. step a) by reacting 3-chloro4-nitroacetanilide with dimethylamine.

Yield: 16.8 g (75.5% of the theoretical) yellow crystals

IR: 3552, 3363 cm-¹ (v CH_{Ar}), 2928, 2801 cm⁻¹ (v CH), 1678 cm⁻¹ (v C═O), 1613 cm⁻¹ (v C═C), 1554 cm⁻¹ (vₐₛ NO₂), 1337 cm⁻¹ (vₛ NO₂).

¹H-NMR: 10.2 ppm (1H, s, NHAc); 7.8 ppm (H³, d, ³J_{H,H}=9.02 Hz); 7.48 ppm (H⁶, d, ⁴J_{H,H}=1.83 Hz); 7.05 ppm (H⁴, dd,³J_{H,H}=9.03 Hz, ⁴J_{H,H}=1.98 Hz); 2.08 ppm (3H, s, C(═O)CH₃); 2.78 ppm (6H, s, N(CH₃)₂).

Step b) N,N-Dimethyl-2-Nitro-5-Aminoaniline

Step b) was carried out in the same as Example 1.3.3. step b) by reacting N,N-dimethyl-2-nitro-5-acetaminoaniline with hydrochloric acid.

Yield: 18.9 g (100% of the theoretical) yellow crystals

IR: 3449, 3337 cm⁻¹ (v CH_{Ar}), 2971, 2872 cm⁻¹ (v CH), 1619 cm⁻¹ (v C═C), 1562 cm⁻¹ (vₐₛ NO₂), 1320 cm⁻¹ (vₛ NO₂).

¹H-NMR: 7.83 ppm (H3, d, ³J_{H,H}=9.70 Hz); 7.29 ppm (2H, s, NH₂); 6.38 ppm (H⁴, dd, ³J_{H,H}=9.76 Hz, ⁴J_{H,H}=2.60 Hz); 6.23 ppm (H⁶, d, ⁴J_{H,H}=2.60 Hz); 3.71 ppm (3H, ³J_{H,H}=4.88 Hz, syn-NCH₃); 3.27 ppm (3H, ³J_{H,H}=4.90 Hz, anti-NCH₃).

1.3.8. Preparation of N,N-Diethyl-2-Nitro-5-Aminoaniline

Step a) N,N-Diethyl-2-Nitro-5-Acetylaminoaniline

Step a) was carried out in the same way as Example 1.3.3. step a) by reacting 3-chloro-4-nitroacetanilide with diethylamine.

Yield: 20.1 g (79.9% of the theoretical) yellow crystals

IR: 3437, 3294 cm⁻¹ (v CH_{Ar}), 2979, 2934 cm⁻¹ (v CH), 1671 cm⁻¹ (v C═O), 1613 cm⁻¹ (v C═C), 1558 cm⁻¹ (vₐₛ NO₂), 1347 cm⁻¹ (vₛ NO₂).

¹H-NMR: 10.43 ppm (1H, s, NHAc); 7.92 ppm (H³, d,³J_{H,H}=9.24 Hz); 7.75 ppm (H⁶, d, ⁴J_{H,H}=2.06 Hz); 7.35 ppm (H⁴, dd, ³J_{H,H}=8.97 Hz, J_{H,H}=2.06 Hz); 3.26 ppm (4H, q, J_{H,H}=7.04 Hz, N(CH₂CH₃)₂); 2.26 ppm (3H, s, C(═O)CH₃); 1.20 ppm (6H, t, ³J_{H,H}=7.04 Hz, N(CH₂CH₃)₂).

Step b) N,N-Diethyl-2-Nitro-5-Aminoaniline

Step b) was carried out in the same way as Example 1.3.3. step b) by reacting N,N-diethyl-2-nitro-5-acetaminoaniline with hydrochloric acid.

Yield: 12.6 g (60.2% of the theoretical)

Melting point: (red oil)

IR: 3469, 3367, 3233 cm⁻¹ (v CH_{Ar}), 2974, 2923, 2872 cm⁻¹ (v CH), 1631 cm⁻¹ (v C═C), 1567 cm⁻¹ (Vₐₛ NO₂), 1344 cm (vₛ NO₂).

¹H-NMR: 7.91 ppm (H³, d, ³J_{H,H}=8.99 Hz); 6.45 ppm (2H, s, NH₂); 6.41 ppm (H⁶, d, ⁴J_{H,H}=2.23 Hz); 6.32 ppm (H⁴, dd,³J_{H,H}=9.08 Hz, ⁴J_{H,H}=2.23 Hz); 3.23 ppm (3H, t, J³_{H,H}=7.04 Hz, N(CH₂-CH₃)₂); 1.21 ppm (3H, t,³J_{H,H}=7.03 Hz, N(CH₂CH₃)₂).

1.3.9. Preparation of N-(5-Amino-2-Nitrophenyl)Pyrrolidine

Step a) N-(5Acetylamino-2-Nitrophenyl)Pyrrolidine

Step a) was carried out in the same way as Example 1.3.3. step a) by reacting 3-chloro-4-nitroacetanilide with pyrrolidine.

Yield: 24.3 g (97.5% of the theoretical) yellow crystals

IR: 3262 cm⁻¹ (v CH_{Ar}), 2969, 2873 cm⁻¹ (v CH), 1667 cm⁻¹(v C═O), 1614 cm⁻¹ (v C═C), 1548 cm⁻¹ (vₐₛ NO₂), 1358 cm⁻¹ (vₛ NO₂).

¹H-NMR: 10.38 ppm (1H, s, NHAc); 7.91 ppm (H³, d, ³J_{H,H}=9.05 Hz); 7.61 ppm (H⁶, d, ⁴J_{H,H}=1.95 Hz); 7.11 ppm (H⁴, dd, ³J_{H,H}=9.06 Hz, ⁴J_{H,H}=2.01 Hz); 3.27 ppm (4H, t, ³J_{H,H}=6.25 Hz, N(CH₂)₂); 2.25 ppm (3H, s, C(═O)CH₃); 2.09 ppm (4H, t, ³J_{H,H}=6.25 Hz, N(CH₂CH₂)₂).

Step b) N-(5-Amino-2-Nitrophenyl)Pyrrolidine

Step b) was carried out in the same way as Example 1.3.3. step b) by reacting N-(5-acetylamino-2-nitrophenyl)pyrrolidine with hydrochloric acid.

Yield: 18.3 g (98.1% of the theoretical)

Melting point: (red oil)

IR: 3468, 3366, 3231 cm⁻¹ (v CH_{Ar}), 2970, 2874 cm⁻¹ (v CH), 1608 cm⁻¹ (v C═C), 1560 cm⁻¹ (vₐₛ NO₂), 1360 cm⁻¹ (vₛ NO₂).

¹H-NMR: 7.81 ppm (H³, d, ³J_{H,H}=9.35 Hz); 6.32 ppm (2H, s, NH₂); 6.18 ppm (H⁴H⁶, m); 3.25 ppm (4H, t, ³J_{H,H}=6.34 Hz, N(CH₂CH₂)₂); 2.05 ppm (4H, t, ³J_{H,H}=6.35 Hz, N(CH₂CH₂)₂).

1.3.10. Preparation of N-(5-Amino-2-Nitrophenyl)Piperidine

Step a) N-(5-Acetylamino-2-Nitrophenyl)Piperidine

Step a) was carried out in the same way as Example 1.3.3. step a) by reacting 3-chloro-4-nitroacetanilide with piperidine.

Yield: 22.1 g (84.1% of the theoretical) yellow crystals

Step b) N-(5-Amino-2-Nitrophenyl)Piperidine

Step b) was carried out in the same way as Example 1.3.3. step b) by reacting N-(5-acetylamino-2-nitrophenyl)piperidine with hydrochloric acid.

Yield: 17.8 g (81.5% of the theoretical)

Melting point: (red oil)

IR: 3449, 3358, 3245 cm⁻¹ (v CH_{Ar}), 2990, 2938 cm⁻¹ (v CH), 1604 cm⁻¹ (v C═C), 1563 cm⁻¹ (vₐₛ NO₂), 1341 cm⁻¹ (vₛ NO₂).

¹H-NMR: 7.83 ppm (H³, d, ³J_{H,H}=8.91 Hz); 6.40 ppm (2H, s, NH₂); 6.19 ppm (H⁴, dd, ³J_{H,H}=4.36 Hz, ⁴J_{H,H}=2.19 Hz); 6.15 ppm (H⁶, d, ⁴J_{H,H}=2.29 Hz); 2.88 ppm (4H, t, ³J_{H,H}=5.37 Hz, N(CH₂—CH₂)₂CH₂); 1.63 ppm (4H, m, N(CH₂CH₂)₂CH₂); 1.54 ppm (2H, m, N(CH₂CH₂)₂CH₂).

1.3.11. Preparation of N-(5-amino-2-Nitrophenyl)Azepan

Step a) N-(5-Acetylamino-2-Nitrophenyl)Azepan

Step a) was carried out in the same way as Example 1.3.3. step a) by reacting 3-chloro-4-nitroacetanilide with azepan.

Yield: 25.3 g (91.3% of the theoretical) yellow crystals

IR: 3331, 3285 cm⁻¹ (v CH_{Ar}), 2934, 2856 cm⁻¹ (v CH), 1700, 1681 cm⁻¹ (v C═O), 1613 cm⁻¹ (v C═C), 1549 cm⁻¹ (vₐₛ NO₂), 1338 cm⁻¹ (vₛ NO₂).

¹H-NMR: 10.2 ppm (1H, s, NHAc); 7.70 ppm (H³, d, ³J_{H,H}=9.02 Hz); 7.62 ppm (H⁶, d, ⁴J_{H,H}=1.85 Hz); 6.97 ppm (H⁴, dd, ³J_{H,H}=8.98 Hz, ⁴J_{H,H}=1.83 Hz); 3.20 ppm (4H, t, ³J_{H,H}=5.57 Hz, N(CH₂)₂); 2.08 ppm (3H, s, C(═O)CH₃); 1.76 ppm (4H, s, N(CH₂CH₂)₂); 1.52 ppm (4H, s, N(CH₂CH₂CH₂)₂).

Step b) N-(5-Amino-2-Nitrophenyl)Azepan

Step b) was carried out in the same way as Example 1.3.3. step b) by reacting N-(5-acetylamino-2-nitrophenyl)azepan with hydrochloric acid.

Yield: 19.8 g (95.6% of the theoretical) yellow crystals

IR: 3460, 3353 cm⁻¹ (v CH_{Ar}), 3230, 2926, 2855 cm⁻¹ (v CH), 1607 cm⁻¹ (v C═C), 1550 cm⁻¹ (vₐₛ NO₂), 1362 cm⁻¹ (vₛ NO₂).

¹H-NMR: 7.65 ppm (H³, d, ³J_{H,H}=9.08 Hz); 6.19 ppm (H⁶, d, ⁴J_{H,H}=1.95 Hz); 6.16 ppm (2H, s, NH₂); 6.05 ppm (H⁴, dd,³J_{H,H}=9.02 Hz, ⁴J_{H,H}=1.85 Hz); 3.18 ppm (4H, t, ³J_{H,H}=5.48 Hz, N(CH₂)₂); 1.72 ppm (4H, s, N(CH₂CH₂)₂); 1.53 ppm (4H, s, N(CH₂CH₂—CH₂)₂).

1.3.12. Preparation of N-(5-Amino-2-Nitrophenyl)Morpholine

Step a) N-(5-Acetylamino-2-Nitrophenyl)Morpholine

Step a) was carried out in the same way as Example 1.3.3. step a) by reacting 3-chloro-4-nitroacetanilide with morpholine.

Yield: 25.6 g (96.5% of the theoretical) yellow crystals

IR: 3291 cm⁻¹ (v CH_{Ar}), 2968, 2836 cm⁻¹ (v CH), 1698 cm⁻¹ (v C═O), 1612 cm⁻¹ (v C═C), 1550 cm⁻¹ (vₐₛ NO₂), 1331 cm⁻¹ (vₛ NO₂).

¹H-NMR: 10.54 ppm (1H, s, NHAc); 8.1 ppm (H³, d, ³J_{H,H}=9.02 Hz); 7.71 ppm (H6, d, ⁴J_{H,H}=2.08 Hz); 7.48 ppm (H⁴, dd, ³J_{H,H}=8.97 Hz, ⁴J_{H,H}=2.08 Hz); 3.9 ppm (4H, t, ³J_{H,H}=4.54 Hz, N(CH₂CH₂O)₂); 3.13 ppm (4H, t, ³J_{H,H}=4.56 Hz, N(CH₂-CH₂O)₂); 2.27 ppm (3H, s, C(═O)CH₃).

Step b) N-(5-Amino-2-Nitrophenyl)Morpholine

Step b) was carried out in the same way as Example 1.3.3. step b) by reacting N-(5-acetylamino-2-nitrophenyl)morpholine with hydrochloric acid.

Yield: 19.2 g (95.6% of the theoretical) yellow crystals

Melting point: (red oil)

IR: 3466, 3320, 3220 cm⁻¹ (v CH_{Ar}), 2970, 2867 cm⁻¹ (v CH), 1606 cm⁻¹ (v C═C), 1572 cm⁻¹ (vₐₛ NO₂), 1344 cm⁻¹ (vₛ NO₂).

¹H-NMR: 7.88 ppm (H³, d, ³J_{H,H}=8.63 Hz); 6.51 ppm (2H, s, NH₂); 6.22 ppm (H⁴H⁶, m); 3.73 ppm (4H, t,³J_{H,H}=4.44 Hz, N(CH₂CH₂—O)₂); 2.92 ppm (4H, t, ³J_{H,H}=4.47 Hz, N(CH₂CH₂O)₂).

1.3.13. Preparation of N-(5-Amino-2-Nitrophenyl)Piperazine

Step a) N-(5-Acetylamino-2-Nitrophenyl)Piperazine

Step a) was carried out in the same way as Example 1.3.3. step a) by reacting 3-chloro-4-nitroacetanilide with piperazine;

Yield: 21.5 g (81 % of the theoretical) yellow crystals

IR: 3435 cm⁻¹ (v CH_{Ar}), 3039, 2920, 2853 cm⁻¹ (v CH), 1695 cm⁻¹ (v C═O), 1615 cm⁻¹ (v C═C), 1558 cm⁻¹ (vₐₛ NO₂), 1366 cm⁻¹ (vₛ NO₂).

¹H-NMR: 10.32 ppm (1H, s, NHAc); 7.86 ppm (H³, d, ³J_{H,H}=9.01 Hz); 7.51 ppm (H⁶, d, ⁴J_{H,H}=1.93 Hz); 7.25 ppm (H⁴, dd, ³J_{H,H}=8.99 Hz, ⁴J_{H,H}=1.97 Hz); 2.86 ppm (4H, t, ³J_{H,H}=5.65 Hz, N(CH₂CH₂NH)₂); 2.82 ppm (4H, t, ³J_{H,H}=5.67 Hz, N(CH₂CH₂—NH)₂); 2.09 ppm (3H, s, C(═O)CH₃).

Step b) N-(5-Amino-2-Nitrophenyl)Piperazine

Step b) was carried out in the same way as Example 1.3.3. step b) by reacting N-(5-acetylamino-2-nitrophenyl)-piperazine with hydrochloric acid.

Yield: 14.6 g (93.8% of the theoretical) yellow crystals

IR: 3411, 3317, 3217 cm⁻¹ (v CH_{Ar}), 2962, 2837 cm⁻¹ (v CH), 1608 cm⁻¹ (v C═C), 1567 cm⁻¹ (vₐₛ NO₂), 1349 cm⁻¹ (vₛ NO₂).

¹H-NMR: 7.88 ppm (H³, d, ³J_{H,H}=9.66 Hz); 6.59 ppm (2H, s, NH₂); 6.26 ppm (H⁴, dd, ³J_{H,H}=9.56 Hz, ⁴J_{H,H}=2.18 Hz); 6.25 ppm (H⁶, d, ⁴J_{H,H}=2.07 Hz); 5.2 ppm (1H, s, NH); 3.02 ppm (8H, s, N(CH₂CH₂NH)₂).

1.4. Other Compounds According to the Invention Corresponding to General Formula (I)

Other compounds according to the invention corresponding to general formula (I) are listed in Table 1 below.

TABLE 1
Compounds according to the invention corresponding to general
formula (I), their absorption maximum and their color

| No. | R¹ | R² | R³ | R⁴ | R⁵ | R⁶ | R⁷ | λₘₐₓ | Color |
|---|---|---|---|---|---|---|---|---|---|
| 1 | H | H | H | H | CH₃ | H | H | 444 | Yel- |
| | | | | | | | | | low |
| 2 | H | H | H | H | H | CH₃ | H | 443 | Yel- |
| | | | | | | | | | low |
| 3 | H | H | H | H | H | H | CH₃ | 440 | Yel- |
| | | | | | | | | | low |
| 4 | H | H | H | H | OCH₃ | H | H | 456 | Yel- |
| | | | | | | | | | low |
| 5 | H | H | H | H | H | OCH₃ | H | 427 | Yel- |
| | | | | | | | | | low |
| 6 | H | H | H | H | H | H | OCH₃ | 444 | Yel- |
| | | | | | | | | | low |
| 7 | H | CH₃ | H | CH₃ | F | H | H | 452 | Yel- |
| | | | | | | | | | low |

2. Coloring
2.1. Creme-based colorant:

| | | |
|---|---|---|
| Dye corresponding to formula (I) | x.x | g |
| Sodium lauryl sulfate (70%) | 2.5 | g |
| Oleic acid | 1.0 | g |
| Sodium sulfite, anhydrous | 0.6 | g |
| Cetostearyl alcohol | 12.0 | g |
| Myristyl alcohol | 6.0 | g |
| Propylene glycol | 1.0 | g |
| Ammonia, 25% | 10.0 | g |
| Oxidation dye precursors | y.y | g |
| Water | to 100 | g |

2.2. Compounds Corresponding to Formula (I)

(I-1) N,N-dimethyl-3-amino-4-nitroaniline

(I-2) N-(3-amino-4-nitrophenyl)-morpholine

(I-3) N-(5-amino-2-nitrophenyl)-pyrrolidine

(I-4) N-(5-amino-2-nitrophenyl)piperidine

(I-5) N-(5-amino-2-nitrophenyl)morpholine

2.3. Oxidation Dye Precursors

Primary intermediate:

(P-1) 2,5-diaminotoluene sulfate

Secondary intermediate:

(S-1) 2-amino-4-hydroxyethylaminoanisole sulfate (HC Blau AC)

2.4. Procedure

50 g of the colorant (in the case of the colorant containing the oxidation dye precursors, mixed just before use with 50 g of a 6% H₂O₂ solution in water) were applied by brush to 100% grey hair (4 g of colorant per g of hair). After a contact time of 30 minutes at room temperature, the colorant was rinsed off and the hair was dried.

2.5. Results

The coloring results are set out in the following Table:

| | Dye of | Oxidation dye | |
|---|---|---|---|
| Example | formula (I) | precursors | Color |
| 1 | 2.23 g I-1 | — | Uniform light orange |
| 2 | 2.21 g I-2 | — | Uniform orange |
| 3 | 2.21 g I-2 | 0.27 g P-1 | |
| | | +0.79 g S-1 | Uniform wheat blond |
| 4 | 2.07 g I-3 | — | Uniform light orange |
| 5 | 2.21 g I-4 | — | Uniform light yellow |
| 6 | 2.23 g I-5 | — | Uniform yellow |
| 7 | 2.23 g I-5 | 0.27 g P-1 | Uniform light violet |
| | | +0.79 g S-1 | with blue reflection |

1. A colorant for coloring keratin fibers, characterized in that it contains as substantive dye a 4-nitroaniline derivative corresponding to formula (I): 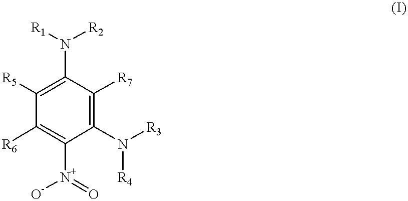
in which R₁, R₂, R₃ and R₄ independently of one another represent a hydrogen atom, an alkyl, hydroxyalkyl, alkoxyalkyl, carbamyl alkyl, mesylaminoalkyl, acetylaminoalkyl, ureidoalkyl, carbalkoxyaminoalkyl, sulfalkyl, piperidinoalkyl, morpholinoalkyl or phenyl radical optionally substituted by an amino group in the para position, these alkyl or alkoxy groups containing 1 to 4 carbon atoms, with the proviso that the four substituents R₁, R₂, R₃ and R₄ do not simultaneously represent hydrogen, and the groups —NR₁R₂ and/or —NR₃R₄ may also stand for an aziridine, acetidine, pyrrolidine, piperidine, azepan, azocine, morpholine, thiomorpholine or piperazine ring which may also bear another substituent R₈ at the nitrogen atom, R₈ being a hydrogen atom, a (C₁₋₄)-alkyl, hydroxy-(C₂₋₃)-alkyl, (C₁₋₄)-alkoxy-(C₂₋₃)-alkyl, amino-(C₂₋₃)-alkyl or 2,3-dihydroxypropyl group, and R₅, R₆ and R₇ independently of one another represent a hydrogen atom, a halogen atom, a (C₁₋₄)-alkyl, (C₁₋₄)-alkoxy, carboxy, sulfo or (C₂₋₄)-hydroxyalkyl group,
or a physiologically compatible salt of these compounds with an inorganic or organic acid.

2. A colorant as claimed in claim 1, characterized in that one of the groups —NR₁R₂ or —NR₃N₄ stands for an aziridine, acetidine, pyrrolidine, piperidine, azepan, azocine, morpholine, thiomorpholine or piperazine ring which may also bear another substituent R₈ at the nitrogen atom, R₈ being a hydrogen atom, a (C₁₋₄)-alkyl, hydroxy-(C₂₋₃)-alkyl, (C₁₋₄)-alkoxy-(C₂₋₃)-alkyl, amino-(C₂₋₃)-alkyl or 2,3-dihydroxypropyl group.

3. A colorant as claimed in claim 1 or 2, characterized in that the compound corresponding to formula (I) is present in quantities of 0.001 to 10% by weight and, more particularly, 0.1 to 5% by weight, based on the colorant as a whole.

4. A colorant as claimed in any of claims 1 to 3, characterized in that it contains another substantive dye.

5. A colorant as claimed in any of claims 1 to 4, characterized in that it additionally contains an oxidation dye precursor of the primary intermediate type.

6. A colorant as claimed in any of claims 1 to 5, characterized in that it additionally contains an oxidation dye precursor of the secondary intermediate type.

7. The use of 4-nitroanilines corresponding to general formula (I) in claim 1 or 2 for coloring keratin fibers.
